(19) United States
(12) Patent Application Publication
(10) Pub. No.: US 2026/0360501 A1
(43) Pub. Date: Nov. 12, 2026

(54) Product, System, and Method for Personalization of Gain and Operation Point in Automatic Insulin Delivery Devices

(71) Applicant: University of Virginia Patent Foundation, Charlottesville, VA (US)

(72) Inventors: Elliott C. Pryor, Helena, MT (US); Marcela Moscoso-Vasquez, Charlottesville, VA (US); Marc D. Breton, Charlottesville, VA (US); Anas El Fathi, Charlottesville, VA (US)

(21) Appl. No.: 19/559,616

(22) Filed: Mar. 6, 2026

Related U.S. Application Data
(60) Provisional application No. 63/801,717, filed on May 7, 2025.

(51) Int. Cl. G16H 20/17; G16H 50/70

(52) U.S. Cl. CPC G16H 20/17; G16H 50/70

(57) ABSTRACT

Quantifiable metrics like TDI allows a method to rescale insulin inputs to match a virtual “nominal” subject, allowing the AID system to operate on standardized parameters. The output is then rescaled to suit the patient's unique insulin requirements. This approach simplifies implementation while achieving personalization, bridging the gap between generalized models and individual specific control.

### CROSS-REFERENCE TO RELATED APPLICATION

This application claims priority to and incorporates by reference U.S. Provisional Patent Application Ser. No. 63/801,717, filed on May 7, 2025, entitled Product, System, and Method for Personalization of Gain and Operation Point in Automatic Insulin Delivery Devices.

### STATEMENT REGARDING FEDERALLY FUNDED RESEARCH

This invention was made with government support under NIH Grant No. DK129553 awarded by National Institutes of Health. The government has certain rights in the invention.

### FIELD

This disclosure includes a specialized piece of software operating in conjunction with an automatic insulin delivery (AID) system as a data input wrapper function to achieve personalization of gain and operation point for using the AID system with an insulin receiving patient.

### BACKGROUND

Over the past few years, automated Insulin Delivery (AID) systems have rapidly revolutionized the management of Type 1 diabetes (T1D). As the vast majority of first and second generation AID systems are based on mathematical models, it can be difficult to account for the large variability in insulin needs between patients. Therefore, many AID systems are optimized for an average patient, with limited adaptation to individual insulin requirements.

A need exists in the art of automated insulin delivery to provide more personalized approaches to using models that tailor medication delivery to a particular patient's needs.

### SUMMARY

Other aspects and features according to the example embodiments of the disclosed technology will become apparent to those of ordinary skill in the art, upon reviewing the following detailed description in conjunction with the accompanying figures. One overall goal is to provide a computer implemented method of determining a Personalized Gain and Operation Point (POGO) for use with an automated insulin delivery system (AID).

In one embodiment, a computer program product for adjusting automated insulin delivery to a recipient individual includes a non-transitory computer usable medium having computer readable program code embodied therein, wherein the computer readable program code comprises instructions for implementing a method with a processor connected to computer memory. The method includes steps of accessing population-level insulin data used by an automated insulin delivery device to determine insulin doses for the recipient individual having an individual insulin dose history; selecting a scaling factor (θ) from the population-level data and calculating, from the population-level data, nominal insulin doses for a nominal individual characterized as a virtual subject in the computer memory; using the scaling factor (θ) to generate a virtual insulin history for the nominal individual and storing the virtual insulin history for the nominal individual in the computer memory; calculating a ratio, gamma (γ), equal to the scaling factor (θ indv) for the recipient individual divided by the scaling factor (θ nominal) for the nominal individual; calculating a standardized insulin dose for the recipient individual by scaling the individual insulin dose history with gamma (γ); applying the standardized insulin dose to the automated insulin delivery device and calculating an insulin dose recommendation for the recipient individual; re-scaling the insulin dose recommendation with the ratio, gamma (γ), and calculating a final dose of insulin delivery to the recipient individual.

A computer implemented method used in the product is also claimed.

### BRIEF DESCRIPTION OF THE DRAWINGS

Reference will now be made to the accompanying drawings, which are not necessarily drawn to scale.

FIG. 1 illustrates a system diagram of modulating insulin values to be equivalent to a nominal subject. The automated insulin delivery (AID) system can then operate on this constant, nominal subject, and then the values are re-scaled for the individual.

FIG. 2 illustrates example continuous glucose monitoring (CGM) traces of a subject with high insulin needs (total daily insulin (TDI)=123). FIG. 2A shows traces using embodiments of this disclosure for personalization of gain and operation point (POGO), and

FIG. 2B shows other traces that were not subject to the functions described herein (no-POGO). The bottom plot shows the insulin infusion requested by the systems. The subjects received same meal and variability inputs, but the AID system was run with and without operations of scaling according to this disclosure

FIG. 3 illustrates example CGM traces of a subject with high insulin needs (TDI=123) using POGO (first traces) and no-POGO (other traces). The bottom plot shows the insulin infusion requested by the POGO and no-POGO systems. The subjects received same meal and variability inputs, but the AID system was run with and without POGO.

FIG. 4 illustrates comparisons of overnight vs daytime traces. FIG. 4A illustrates Time Below Range (% time <70 mg/dL), and FIG. 4B illustrates Time in Tight Range (% time in 70-140 mg/dL), both of which are presented vs the total daily insulin dosed by the system during daytime operation. FIG. 4C illustrates Time Below Range (% time <70 mg/dL), and FIG. 4D illustrates Time in Tight Range (% time in 70-140 mg/dL), both of which are presented vs the total daily insulin dosed by the system during overnight operation. The figures provide a comparison of AID performance metrics with (in red) and no-POGO (blue). The shaded region shows the 25-75 percentiles, and the solid line indicates the median value within a sliding window is a schematic of a computer environment used in accordance with the disclosure herein.

FIG. 5 is a schematic of a computer environment used in accordance with the disclosure herein.

FIG. 6 is a schematic of a computer environment used in accordance with the disclosure herein.

### DETAILED DESCRIPTION

Over the past few years, Automated Insulin Delivery (AID) systems have rapidly revolutionized the management of Type 1 diabetes (T1D). As the vast majority of first and second generation AID systems are based on mathematical models, it can be difficult to account for the large variability in insulin needs between patients. Therefore, many AID systems are optimized for an average patient, with limited adaptation to individual insulin requirements. This disclosure proposes a specialized piece of software operating in conjunction with an AID system as a data input wrapper function to achieve Personalization Of Gain and Operation Point (referred to herein by the acronym POGO). In non-limiting embodiments, this disclosure adjusts an AID system to the insulin needs of any user, by rescaling of the insulin inputs to an AID device, and configuring a controller in the AID device to provide outputs that are linearly based on a ratio of insulin needs. This disclosure demonstrates improvement, in silico, in model prediction and glycemic control performances across a wide range of insulin needs while avoiding the need for complex personalization.

This disclosure discusses how devices implementing automatic insulin delivery (AID) to recipient individuals (i.e., patients using the AID devices) may use controllers, control data, algorithms, and parameters that have been configured with large sample data sets and test data sets gathered from larger sets of a population. The population may be insulin receiving patients from a similar region, similar demographic, similar hospital, or other criteria used to gather data sets from an overall population. The data sets that represent large groups of patients are referred to herein as being determined at a “population level” and the relevant population may be determined by an AID device manufacturer. The term “population level” therefore is meant to refer to data, set points and other computer settings that have been gleaned from a selected population of patients that have been subject to testing.

The term “individual” in this disclosure includes all forms of living beings that can utilize automatic insulin delivery, and the term is broad enough to encompass humans and non-human animal recipients of automatic insulin delivery.

Automated insulin delivery (AID) systems have become an increasingly common tool in managing type 1 diabetes (T1D) and are now recommended as standard of care (Forlenza and Lal, 2022; Biester et al., 2024; ADA, 2024). These systems link a continuous glucose monitor (CGM) and an insulin delivery pump via an automated dosing algorithm and adjust insulin delivery every 5 -15 minutes to regulate blood glucose levels. By automating insulin dosing decisions, AID systems reduce the burden of manual glucose management while aiming to improve glycemic control and patient outcomes. As a result, AID systems have become a cornerstone of modern T1D care, offering enhanced convenience and better glycemic outcomes.

However, a key challenge lies in addressing the variability in insulin needs across individuals, which is a limiting factor to the performance of open-loop and closed-loop insulin therapies (Hinshaw et al., 2013). Total Daily Insulin (TDI), a common metric used to quantify an individual's insulin requirements, can vary widely. For example, adults with Type 1 Diabetes (T 1D) may require less than 20 units per day, while others may need well over 100 units, depending on factors such as body weight, age, eating habits, and insulin sensitivity (Wiegand et al., 2008). Although current technologies support model personalization or identification (Gondhalekar et al., 2016; Garcia-Tirado et al., 2021; Villa-Tamayo et al., 2022), practically applying model identification in AID systems may be challenging. Some models may not be identifiable from data (Hoyos et al., 2021; Villa-Tamayo et al., 2022), and running identification on an embedded system may strain computational resources, while running in the cloud requires significant infrastructure.

As a result, most commercial AID systems are designed at the population level, largely relying on generalized models to represent an “average” user (Forlenza and Lal, 2022). These models are attractive because they reduce the complexity of system design and implementation, requiring less effort to deploy across a broad user base. By assuming a middle ground for parameters such as insulin sensitivity, population-level systems can function adequately for users whose needs align with these averages. However, for individuals with insulin needs that significantly deviate from the norm, these systems can underperform.

One standard personalization scheme without model identification is setting the operating point, or basal rate, of the AID system based on individual insulin needs (Messer and Breton, 2023). However, this fails to fully address variations in individual insulin responses away from the operation point. It does not consider how a person's reaction to a bolus might depend on their specific insulin requirements, leading to potential mismatches in control. As such, controller gains or regularization parameters, such as R in MPC cost functions, also need to be personalized based on insulin needs.

Model identification and personalized tuning strategies have been proposed in the literature (Gondhalekar et al., 2016; Shi et al., 2019; Resalat et al., 2019; Garcia-Tirado et al., 2021; Moscoso-Vasquez et al., 2023). However, these are often seen as separate tasks, and they may be nontrivial to implement in practice. The proposed method, Personalization Of Gain and Operation Point (POGO), offers a simple yet effective way to tailor any model-based AID systems to individual insulin needs without complex model identification or individualized controller tuning.

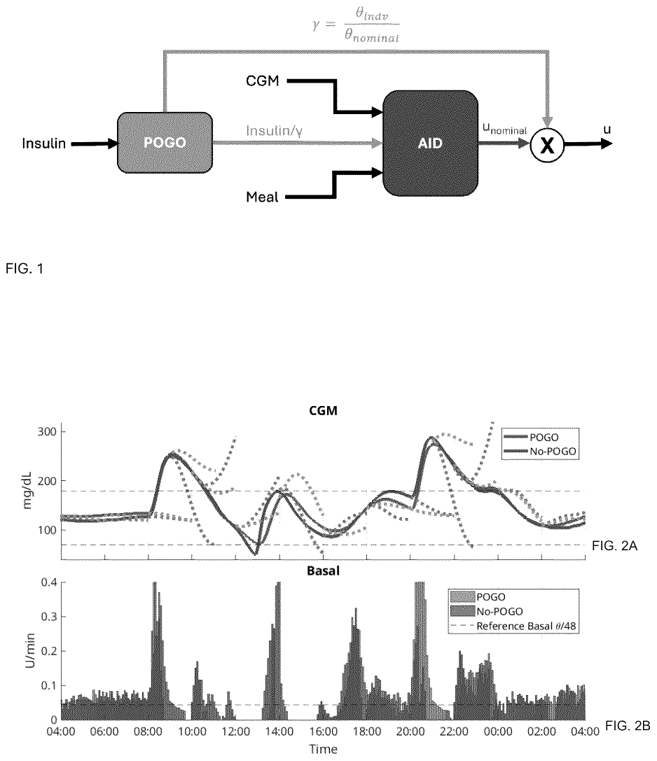

The proposed method, POGO, may be described in one non-limiting embodiment as a wrapper around the population-level AID system. FIG. 1 shows a block diagram of how POGO can be applied to an AID system. Using easily quantifiable metrics like total daily insulin (TDI), the computer program (POGO) rescales insulin inputs to match a virtual “nominal” subject, allowing the AID system to operate on standardized parameters. The output is then rescaled to suit the patient's unique insulin requirements. This approach simplifies implementation while achieving personalization, bridging the gap between generalized models and individual specific control.

One aspect of this disclosure uses a non-limiting theory that an individual insulin response is linear with relative insulin needs among other individuals having a given insulin receiving history. For example, if an individual, A, needs twice as much insulin as a different individual, B, then 2u of insulin in individual A has the same effect as 1u of insulin in individual B. Based on this, insulin inputs to the control system of an AID device can be modified (scaled) to be equivalent to a “nominal” individual that is identified and characterized as a virtual subject having an insulin dosage history that can be generated and saved in a computer memory. This generates an equivalent insulin history for the nominal subject which is then used as input to the AID algorithm of an AID device to generate the insulin dose recommendation, u-nominal. Rescaling the insulin inputs standardizes how the AID system processes data, ensuring consistency across individuals and potentially improving model fit. Then the insulin output can be transformed back to match the individual subject's insulin needs. This allows for seamless personalization of the operation point and gain of the control system, and it can improve model fit while avoiding the need for model personalization. The concept that individual insulin response scales linearly with relative insulin needs of others is consistent with established diabetes care guidelines. For instance, common rules like the 50-50 split between basal and bolus insulin, the 1800 rule for calculating correction factors (CF), and the 450 rule for carbohydrate ratios (CR) inherently reflect linear relationships with Total Daily Insulin (TDI) (King et al., 2016).

For purposes of illustration in a mathematically proven description, let γ be the ratio of insulin needs of a specific person compared to the nominal one, when the insulin history of the nominal individual has been created in silico, based on a selected parameter used in AID algorithms. This ratio, γ, can be estimated using correction factor (CF), total daily insulin (TDI), model insulin sensitivity factor (Si), or another factor (θ). The idea here is that this disclosure can populate an insulin history in computer memory for a nominal individual (i.e., a virtual subject) by selecting an AID parameter used by an AID controller and an AID algorithm, and then adjusting a current patient's insulin doses by scaling. Along these lines, one ration of interest is γ=θ-indv divided by θ-nominal is the ratio of relative insulin needs of an insulin receiving individual compared to the nominal subject. Insulin history of the individual can then be scaled by [INS]i=INSi/γ. The output then gets re-scaled to the right level for the currently receiving individual by u-nominal*γ=u.

For example, in one non-limiting example, this disclosure utilizes a parameter of total daily insulin (TDI) to identify a nominal subject with a Total Daily Insulin (TDI) of 40 units, while a current insulin receiving individual has a TDI of 120 units and received a 6 unit bolus. For this scenario, using TDI as the selected scaling factor, the standardized insulin dose is [INS]i=6/3=2 units. The down-scaling is helpful for accurate system performance, as the raw value of 6 units of insulin might lead to a significant glucose drop in the internal model of the nominal subject, prompting overly cautious control actions. While, for the actual individual, a 6-unit bolus may not have such a profound effect.

This scaling ensures the system interprets the insulin history correctly, potentially improving the model fit, and generate more adequate insulin dose recommendations, u nominal. Similarly, by rescaling AID system outputs, individuals who are more insulin-resistant receive appropriately higher doses through rescaling, while insulin-sensitive individuals are given smaller doses: avoiding the need to personalize the controller tuning parameters.

Examples of this disclosure are included herein. The University of Virginia (UVA) Model Predictive Control (MPC) system was selected as the AID platform for evaluating POGO (Garcia-Tirado et al., 2021; Moscoso-Vasquez et al., 2023). This system is a state-of-the-art fully-closed loop system, designed to operate without meal announcements. This system has been validated in multiple clinical trials, demonstrating its effectiveness in maintaining glycemic control under diverse conditions (Garcia-Tirado et al., 2023; Moscoso-Vasquez et al., 2024; Kovatchev et al., 2024). It is primarily driven by a Model Predictive Control (MPC) algorithm that uses a population-level glucose-insulin model as its basis. The model is based on the Subcutaneous Oral Glucose Minimal Model (SOGMM), and is augmented with a disturbance signal (mg/dL/min) to capture unmodeled phenomenon. For more details, the reader is referred to Patek et al. (2016) and Garcia-Tirado et al. (2021).

The control system includes personalized tuning mechanisms that adjust based on estimated TDI to enable some personalization (Moscoso-Vasquez et al., 2023). These mechanisms enable the controller to modify its operations according to an individual's estimated insulin needs. The operation point (basal rate) is set as ub=TDI/48 (u/h) according to the 50-50 rule, which remains at the nominal value in the POGO system.

The cost function parameters Q(IOB or insulin on board), R(glucose G, dG/dt) are time varying functions that de-tune in response to system state. The Q function penalizes deviation from target glucose; it is designed to be large near basal to promote aggressive meal response and then de-tune in response to IOB to prevent insulin stacking. The R function regularizes control actions to be near to basal; it is designed such that when glucose is stable and in euglycemic range the system is regularized towards the basal rate, but when glucose is rapidly changing or out of euglycemia the system has more freedom to move away from basal rate. Both Q and R functions are parameterized by TDI, which is an example of a personalized tuning strategy. The Q function de-tunes at larger values of TDI and R is strictly smaller when TDI is high. This allows more aggressive control during postprandial period (Q), and generally larger deviations from basal (R). As a result, it is designed to account for some variation in the subject's insulin needs (Moscoso-Vasquez et al., 2023). Using POGO, these functions are still time-varying but do not depend upon the subject's TDI and use the nominal value θ-nominal.

In one non-limiting example Methods and systems of this disclosure may be set to operate with a nominal θ-nominal=40. This roughly represents the average TDI of the population from clinical trials of the algorithm (Moscoso-Vasquez et al., 2024) and that of the UVA/Padova virtual patient population used for controller design (Visentin et al., 2018).

As a linear AID system, one must account for potential operation point errors, both with and without POGO, so a run-to-run methodology is applied to find the θ value that achieves specified target metrics. The aggressiveness of the controller, modulated through θ≈TDI, is adjusted based on glycemic metrics calculated from the last several days of CGM records. In this regard, a modulation factor (α_{θ}) is determined daily based on glycemic metrics, time below range (TBR, % time below 70 mg/dL), time above range (TAR, % time above 180 mg/dL), and the number of hypoglycemia treatments, which are used to compute a penalty to increase TBR. Then alpha-theta is used to modify the tuning parameter theta (which also controls the basal rate ub) by: theta′=alpha x theta x theta(Diaz et al., In press).

### Examples

Simulations were conducted using the University of Virginia (UVA)/Padova Type 1 Diabetes Simulator (Visentin et al., 2018) with 100 adult virtual subjects and the UVA MPC closed-loop system. Three meals per day were included with meal-size randomly selected from normal distributions with 59 +/−9.75 g for breakfast, 61 +/−14.25 g for lunch and 75 +/−12.75 g for dinner. Meal times were randomly selected from normal distributions with 7.5 +/−0.5 h for breakfast, 12.4 +/−0.51 h for lunch; and 19.5 +/−0.95 h for dinner. Fifteen grams (15 g) of hypoglycemia treatments are provided if glucose drops below 60 mg/dL.

To explore a wider range of insulin needs, the nominal insulin sensitivity value of the virtual subject is modulated through various test parameters used in the art, i.e. Kp3, VmX, and Vm0, which modify insulin dependent glucose utilization and endogenous glucose production. The nominal values for each virtual subject are multiplied by {¼, ½, 1, 2, and 4} generating a 500-subject in-silico population, and the operation point was assessed using a run-to-run adaptation strategy.

The operation points for both POGO and Non-POGO methodologies are optimized during a 15-day run-in period using the adaptation scheme defined in Section 2.2. Then, the same 15-day evaluation scenario is repeated using the optimized θ value.

From the simulation scenario, several metrics are collected to evaluate and compare the performance of the AID systems with and without POGO. Control performance is assessed using standard glucose control metrics: TBR, time in range (TIR, % time within 70-180 mg/dL), and TAR. These metrics provided insight into the system's ability to maintain glucose levels within the target range while minimizing hypoglycemia and hyperglycemia. To evaluate improvements in model fit, the root-mean-square error (RMSE) between predicted and observed glucose levels is calculated. The prediction horizon is 2 hours (24 samples) which is that of the UVA MPC. Additionally, the magnitude of estimated disturbance (d(t)) was analyzed to determine how well the system adapted to variations in insulin requirements. Since the system is designed to operate mainly in FCL without any meal-related inputs, the main component for the model-subject mismatch, d(t), should be primarily meal-related.

FIG. 2 compares glucose levels for a subject with high insulin needs, using POGO and no-POGO. Without POGO, the system predicts extreme hyper-and hypoglycemic excursions, while POGO accurately forecasts glucose levels by properly interpreting insulin history. The no-POGO system shows a more pulsatile insulin delivery, with prolonged suspensions, especially in the evening. In contrast, POGO returns to the basal rate due to improved predictions and maintaining the correct Q and R balance in the MPC cost function.

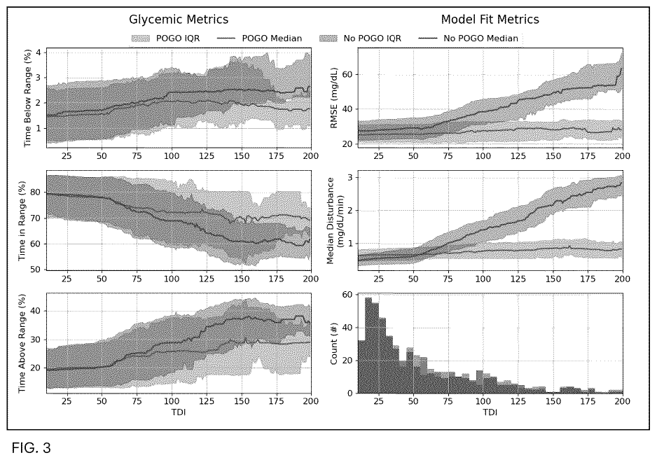

FIG. 3 shows a comparison of the selected performance metrics vs. insulin needs. The simulation results demonstrate significant performance improvements with the integration of POGO into the AID system, especially for individuals with high insulin resistance. Large improvements in TAR were achieved for individuals with high insulin needs, with a maximum improvement of 27.8%, reflecting better control of hyperglycemia. In addition, TBR was slightly reduced or maintained, indicating that these improvements in hyperglycemia management do not increase hypoglycemia exposure. The root-mean-square error (RMSE) between predicted and observed glucose levels decreased significantly across the spectrum of TDI values, highlighting improvements in the system's ability to accurately model individual glucose-insulin dynamics. This reduction was particularly pronounced for subjects with very high or very low TDI values, where the baseline AID system typically struggles due to the limitations of population-level models.

Another observation is the flattening of the disturbance state, which captures deviations from expected glucose behavior. Without POGO, disturbances were closely tied to insulin resistance, with highly resistant individuals exhibiting large d(t) magnitudes. This is caused by d(t) capturing insulin-sensitivity related information, to compensate the difference in the observed glucose value for a given insulin concentration. With POGO, disturbance values became more uniform across all TDI values, indicating that its estimation is no longer driven by the insulin sensitivity of the subject, but rather reflected meal disturbances which are constant size for all subjects. This highlights how the system's ability to handle variability in insulin sensitivity has dramatically improved.

One key point across all metrics is that system performance using POGO is nearly constant with respect to individual insulin needs: which is the goal of POGO. In particular, the model fit indices, RMSE and disturbance, are nearly flat with respect to TDI. This highlights POGO's ability to address the model identification problem as the model fits equally well for all individuals.

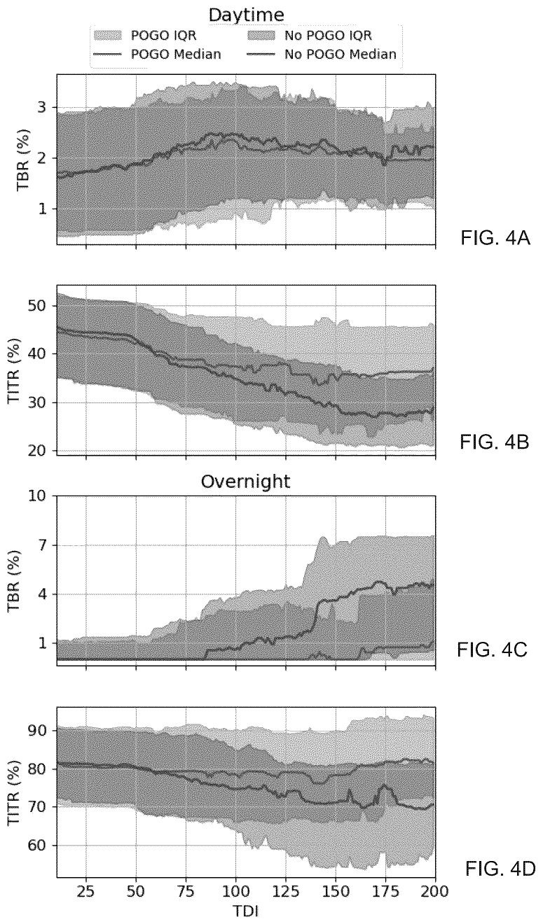

FIG. 4 compares metrics during the daytime and overnight periods, defining the latter as midnight-6am. Reduced TBR can be observed during the overnight period and improved TITR with POGO during both daytime and overnight periods. The increased TBR for the no-POGO system can be explained by a larger-than-required operation point determined by the adaptation system to compensate for daytime hyperglycemia. This results in an overly-aggressive control that, in the absence of disturbances generates significant hypoglycemia. On the other hand, with the POGO system's ability to scale the controller gain, this overcompensation via the operating point disappears, providing improved overnight control. Using POGO resulted in notable improvements in model fit, addressing one of the critical challenges in managing variability in insulin needs. By rescaling inputs and outputs to account for individual insulin requirements, POGO effectively normalized system performance across a wide range of insulin sensitivities, as reflected in the uniformity of metrics like RMSE and disturbance across TDI. This uniform performance was achieved without the need for complex model identification processes. POGO also directly improved glycemic outcomes through the model fit and improved balance of tuning parameters. Subjects with high insulin needs, who typically experience poor control with the population-level model, demonstrated substantial gains in TIR without increasing TBR.

The difference between POGO and non-POGO systems was not very evident in individuals with lower insulin needs. This is perhaps due to the adaptive-tuning measures being more effective for more sensitive individuals. Model RMSE is slightly reduced, using POGO, in insulin-sensitive individuals implying that model-fit is marginally better.

Overall, POGO does not degrade system performance for more sensitive individuals, and it significantly improves performance for individuals with large insulin needs. Designed as a wrapper around existing AID systems, POGO minimizes implementation barriers by requiring no fundamental changes to the underlying control algorithm. Instead, it pre-processes insulin inputs to align with a virtual “nominal” subject and post-processes outputs to match the individual's insulin requirements. This modular approach makes POGO highly versatile, as it can be applied to a wide range of AID systems with minimal modifications.

There are some engineering advantages of considering this personalization based on insulin needs externally as a wrapper rather than internally adjusting models or parameters. First, it can be applied to non model-based control laws such as PID, fuzzy logic, or reinforcement learning. Second, POGO allows the controller to be defined with a constant model. This method can be similar, or even equivalent, to identifying the insulin sensitivity parameter in a model-based approach as done by Heusden et al. (2012). However, some models have identifiability issues (Hoyos et al., 2021; Villa-Tamayo et al., 2022) and loading individual matrices can have deployment and security issues. In addition, neural networks have been proposed for practical implementation of MPC control (Castillo et al., 2023; Kovatchev et al., 2024), and it may be challenging to replicate an MPC law using a non-constant model.

Third, POGO allows for a constant balance of control and tuning parameters. This avoids the need to rebalance them dynamically based on insulin needs. Finally, it can help constrain input bounds for edge-case analysis. POGO constrains the AID system to operate around a constant set of insulin needs, which greatly constrains the viable insulin input domain. This is of particular benefit when validating neural networks, or black box architectures. Using POGO guarantees that, in identical situations, individuals with greater insulin needs receive larger doses, which is not necessarily enforced by black-box non-linear control algorithms.

In this work, TDI was used as a measure of relative insulin needs; however, this relies on the assumption that TDI is determined by individual metabolism and is unrelated to behavioral factors such as meal amounts. Other measures in determining insulin needs could be used that are more robust to behavioral factors, such as individual CF values or by identifying insulin sensitivity based on a model.

Future work can involve demonstration of functionality for multiple AID systems to show the generalizability of the method. POGO can also be compared with model identification methods, to evaluate its performance and efficacy. Further, POGO offers a very simple solution for adapting AID systems to highly concentrated, or more dilute, insulins by simply changing the γ parameter. This could offer increased flexibility and robustness in AID use with alternate insulin formulations.

Automated Insulin Delivery (AID) systems have become a cornerstone of modern type 1 diabetes (T1D) care, offering enhanced convenience and better glycemic outcomes. However, it can be challenging to account for the wide variety in insulin needs across individuals. Model identification and controller re-tuning have been proposed to account for this problem, however, these solutions are non-trivial and may be difficult to deploy in practice. POGO is a simple method designed to simultaneously account for model-mismatch and system retuning for individuals with different insulin needs, by linearly rescaling of the insulin inputs and the controller output based on a ratio of insulin needs.

The results demonstrate significant improvements in model fit and system performance (particularly TIR) for highly insulin-resistant individuals, without compromising control for highly sensitive ones. This highlights the improvements of POGO in the system's ability to accurately model individual glucose-insulin dynamics and improved balance of tuning parameters.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. All references cited and discussed in this specification are incorporated herein by reference in their entireties and to the same extent as if each reference was individually incorporated by reference.

In non-limiting embodiments, a computer program product for adjusting automated insulin delivery to a recipient individual incudes a non-transitory computer usable medium having computer readable program code embodied therein, wherein the computer readable program code comprises instructions for implementing a method with a processor connected to computer memory, the method comprising steps of accessing population-level insulin data used by an automated insulin delivery device to determine insulin doses for the recipient individual having an individual insulin dose history; selecting a scaling factor (θ) from the population-level data and calculating, from the population-level data, nominal insulin doses for a nominal individual characterized as a virtual subject in the computer memory; using the scaling factor (θ) to generate a virtual insulin history for the nominal individual and storing the virtual insulin history for the nominal individual in the computer memory; calculating a ratio, gamma (γ), equal to the scaling factor (θ indv) for the recipient individual divided by the scaling factor (θ nominal) for the nominal individual; calculating a standardized insulin dose for the recipient individual by scaling the individual insulin dose history with gamma (γ); applying the standardized insulin dose to the automated insulin delivery device and calculating an insulin dose recommendation for the recipient individual; re-scaling the insulin dose recommendation with the ratio, gamma (γ), and calculating a final dose of insulin delivery to the recipient individual.

In some embodiments, the ratio, gamma (γ), corresponds to relative insulin needs of the recipient individual compared to the nominal individual.

In some embodiments, the relative insulin needs scale linearly between the recipient individual and the nominal individual.

In the following description, references are made to the accompanying drawings that form a part hereof and that show, by way of illustration, specific embodiments or examples. In referring to the drawings, like numerals represent like elements throughout the several figures.

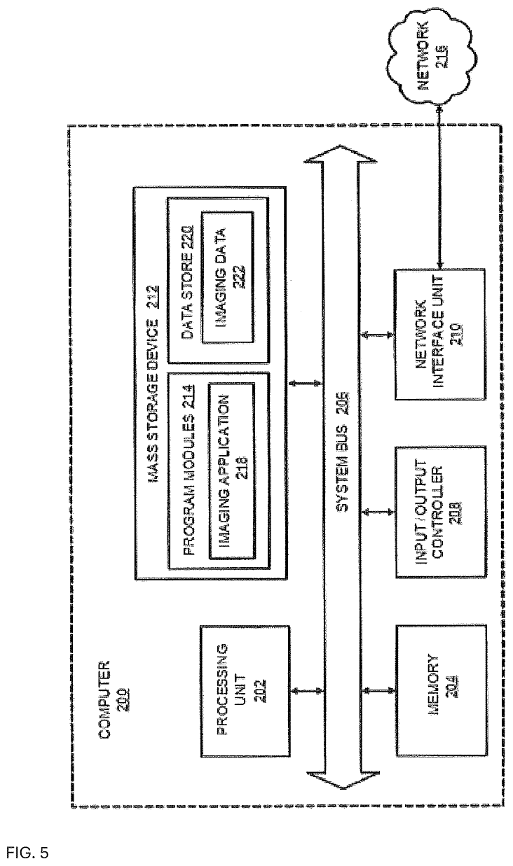

FIG. 5 is a computer architecture diagram showing a general computing system capable of implementing aspects of the present disclosure in accordance with one or more embodiments described herein. A computer 200 may be configured to perform one or more functions associated with embodiments of this disclosure. For example, the computer 200 may be configured to perform operations of the method as described below. It should be appreciated that the computer 200 may be implemented within a single computing device or a computing system formed with multiple connected computing devices. The computer 200 may be configured to perform various distributed computing tasks, which may distribute processing and/or storage resources among the multiple devices. The data acquisition and display computer 150 and/or operator console 110 of the system shown in FIG. 1 may include one or more systems and components of the computer 200.

As shown, the computer 200 includes a processing unit 202 (“CPU”), a system memory 204, and a system bus 206 that couples the memory 204 to the CPU 202. The computer 200 further includes a mass storage device 212 for storing program modules 214. The program modules 214 may be operable to perform one or more functions associated with embodiments of method as illustrated in one or more of the figures of this disclosure, for example to cause the computer 200 to perform operations of the automated DENSE analysis as described below. The program modules 214 may include an imaging application 218 for performing data acquisition functions as described herein, for example to receive image data corresponding to magnetic resonance imaging of an area of interest. The computer 200 can include a data store 220 for storing data that may include imaging-related data 222 such as acquired image data, and a modeling data store 224 for storing image modeling data, or other various types of data utilized in practicing aspects of the present disclosure.

The mass storage device 212 is connected to the CPU 202 through a mass storage controller (not shown) connected to the bus 206. The mass storage device 212 and its associated computer-storage media provide non-volatile storage for the computer 200. Although the description of computer-storage media contained herein refers to a mass storage device, such as a hard disk or CD-ROM drive, it should be appreciated by those skilled in the art that computer-storage media can be any available computer storage media that can be accessed by the computer 200.

By way of example, and not limitation, computer-storage media (also referred to herein as a “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 200. Transitory signals are not “computer-storage media”, “computer-readable storage medium” or “computer-readable storage media” as described herein.

According to various embodiments, the computer 200 may operate in a networked environment using connections to other local or remote computers through a network 216 via a network interface unit 210 connected to the bus 206. The network interface unit 210 may facilitate connection of the computing device inputs and outputs to one or more suitable networks and/or connections such as a local area network (LAN), a wide area network (WAN), the Internet, a cellular network, a radio frequency network, a Bluetooth-enabled network, a Wi-Fi enabled network, a satellite-based network, or other wired and/or wireless networks for communication with external devices and/or systems. The computer 200 may also include an input/output controller 208 for receiving and processing input from a number of input devices. Input devices may include one or more of keyboards, mice, stylus, touchscreens, microphones, audio capturing devices, or image/video capturing devices. An end user may utilize such input devices to interact with a user interface, for example a graphical user interface, for managing various functions performed by the computer 200.

The bus 206 may enable the processing unit 202 to read code and/or data to/from the mass storage device 212 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer-storage media may also represent secondary storage, whether implemented as hard drives or otherwise. Hard drive implementations may be characterized as solid state or may include rotating media storing magnetically-encoded information. The program modules 214, which include the imaging application 218, may include instructions that, when loaded into the processing unit 202 and executed, cause the computer 200 to provide functions associated with embodiments illustrated herein. The program modules 214 may also provide various tools or techniques by which the computer 200 may participate within the overall systems or operating environments using the components, flows, and data structures discussed throughout this description.

In general, the program modules 214 may, when loaded into the processing unit 202 and executed, transform the processing unit 202 and the overall computer 200 from a general-purpose computing system into a special-purpose computing system. The processing unit 202 may be constructed from any number of transistors or other discrete circuit elements, which may individually or collectively assume any number of states. More specifically, the processing unit 202 may operate as a finite-state machine, in response to executable instructions contained within the program modules 214. These computer-executable instructions may transform the processing unit 202 by specifying how the processing unit 202 transitions between states, thereby transforming the transistors or other discrete hardware elements constituting the processing unit 202.

Encoding the program modules 214 may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer-storage media are implemented as semiconductor-based memory, the program modules 214 may transform the physical state of the semiconductor memory, when the software is encoded therein. For example, the program modules 214 may transform the state of transistors, capacitors, or other discrete circuit elements constituting the semiconductor memory.

As another example, the computer-storage media may be implemented using magnetic or optical technology. In such implementations, the program modules 214 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 300) and software architectures that can be deployed in example embodiments.

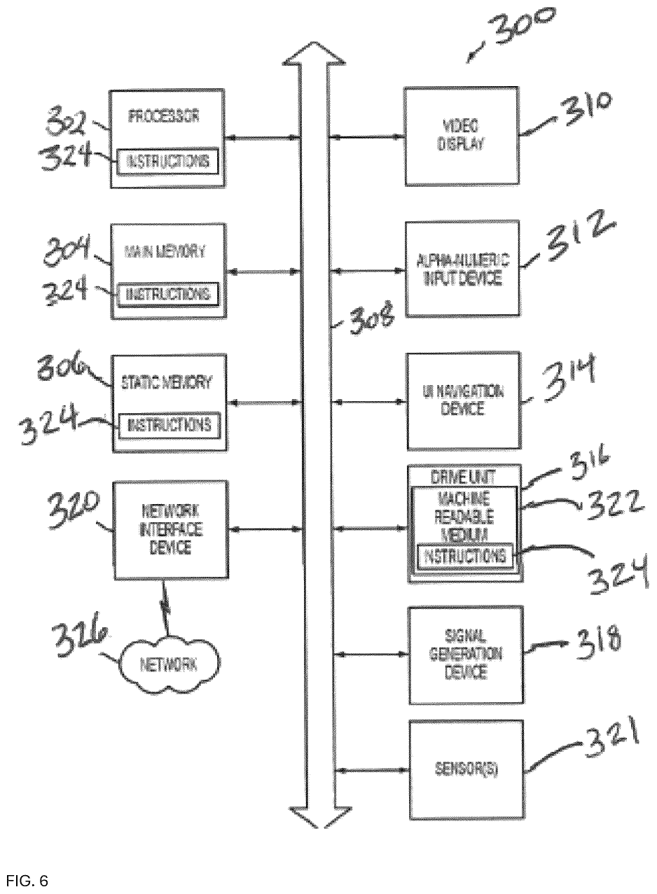

The machine 300 of FIG. 6 can operate as a standalone device or the machine 300 can be connected (e.g., networked) to other machines. In a networked deployment, the machine 300 can operate in the capacity of either a server or a client machine in server-client network environments. In an example, machine 300 can act as a peer machine in peer-to-peer (or other distributed) network environments. The machine 300 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 300. Further, while only a single machine 300 is illustrated, the term “machine” shall also be taken to include any collection of machines that individually or jointly execute a set (or multiple sets) of instructions to perform any one or more of the methodologies discussed herein.

Example machine (e.g., computer system) 300 can include a processor 302 (e.g., a central processing unit (CPU), a graphics processing unit (GPU) or both), a main memory 304 and a static memory 306, some or all of which can communicate with each other via a bus 308. The machine 300 can further include a display unit 310, an alphanumeric input device 312 (e.g., a keyboard), and a user interface (UI) navigation device 311 (e.g., a mouse). In an example, the display unit 810, input device 317 and UI navigation device 314 can be a touch screen display. The machine 300 can additionally include a storage device (e.g., drive unit) 316, a signal generation device 318 (e.g., a speaker), a network interface device 320, and one or more sensors 321, such as a global positioning system (GPS) sensor, compass, accelerometer, or other sensor. The storage device 316 can include a machine readable medium 322 on which is stored one or more sets of data structures or instructions 324 (e.g., software) embodying or utilized by any one or more of the methodologies or functions described herein. The instructions 324 can also reside, completely or at least partially, within the main memory 304, within static memory 306, or within the processor 302 during execution thereof by the machine 300. In an example, one or any combination of the processor 302, the main memory 304, the static memory 306, or the storage device 316 can constitute machine readable media. While the machine readable medium 322 is illustrated as a single medium, the term “machine readable medium” can include a single medium or multiple media (e.g., a centralized or distributed database, and/or associated caches and servers) that configured to store the one or more instructions 324. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media.

Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magnetooptical disks; and CD-ROM and DVD-ROM disks. The instructions 324 can further be transmitted or received over a communications network 326 using a transmission medium via the network interface device 320 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### References

American Diabetes Association (2024). 7. Diabetes Technology: Standards of care in diabetes—2025. Diabetes Care, 48(Supplement1), S146-S166.

Biester, T., Berget, C., Boughton, C., Cudizio, L., Ekhlaspour, L., Hilliard, M. E., Reddy, L., Sap Ngo Um, S., Schoelwer, M., Sherr, J. L., and Dovc, K. (2024). Ispad clinical practice consensus guidelines 2024: Diabetes technologies: Insulin delivery. Hormone Research in Paediatrics, 1-34.

Castillo, A., Villa-Tamayo, M. F., Pryor, E., Garcia-Tirado, J., Colmegna, P., and Breton, M. (2023). Deep neural network architectures for an embedded MPC implementation: Application to an Automated Insulin Delivery system.

In IFAC-PapersOnLine, volume 56, 11521-11526. Elsevier B. V. Diaz, J., Colmegna, P., Pryor, E., and Breton, M. D. (In press). A performance-based adaptation index for automated insulin delivery systems. Journal of Diabetes Science and Technology.

Forlenza, G. P. and Lal, R. A. (2022). Current status and Emerging options for Automated Insulin Delivery Systems. Diabetes technology & therapeutics, 24(5), 362-371.

Garcia-Tirado, J., Colmegna, P., Villard, O., Diaz, J. L., Esquivel-Zuniga, R., Koravi, C. L., Barnett, C. L., Oliveri, M. C., Fuller, M., Brown, S. A., Deboer, M. D., and Breton, M. D. (2023). Assessment of meal anticipation for improving fully automated insulin delivery in adults with type 1 diabetes. Diabetes Care, 46, 1652-1658. DCLP6.

Garcia-Tirado, J., Lv, D., Corbett, J. P., Colmegna, P., and Breton, M. D. (2021). Advanced hybrid artificial pancreas system improves on unannounced meal response—in silico comparison to currently available system. Computer Methods and Programs in Biomedicine, 211.

Gondhalekar, R., Dassau, E., and Doyle, F. J. (2016). Periodic zone-mpc with asymmetric costs for outpatient-ready safety of an artificial pancreas to treat type 1 diabetes. Automatica, 71, 237-246.

Heusden, K. V., Dassau, E., Zisser, H. C., Seborg, D. E., and Doyle, F. J. (2012). Control-relevant models for glucose control using a priori patient characteristics. IEEE Transactions on Biomedical Engineering, 59, 1839-1849.

Hinshaw, L., Dalla Man, C., Nandy, D. K., Saad, A., Bharucha, A. E., Levine, J. A., Rizza, R. A., Basu, R., Carter, R. E., Cobelli, C., et al. (2013). Diurnal pattern of insulin action in type 1 diabetes: implications for a closed loop system. Diabetes, 62(7), 2223-2229.

Hoyos, J. D., Villa-Tamayo, M. F., Builes-Montano, C. E., Ramirez-Rincon, A., Godoy, J. L., Garcia-Tirado, J., and Rivadeneira, P. S. (2021). Identifiability of control-oriented glucose-insulin linear models: Review and analysis. IEEE Access, 9, 69173-69188.

King, A. B., Kuroda, A., Matsuhisa, M., and Hobbs, T. (2016). A review of insulin-dosing formulas for continuous subcutaneous insulin infusion (csii) for adults with type 1 diabetes. Current diabetes reports, 16, 1-8.

Kovatchev, B., Castillo, A., Pryor, E., Kollar, L. L., Barnett, C. L., DeBoer, M. D., and Brown, S. A. (2024). Neural-net artificial pancreas: A randomized crossover trial of a first-in-class automated insulin delivery algorithm. Diabetes Technology and Therapeutics, 26, 375-382.

Messer, L. H. and Breton, M. D. (2023). Therapy settings associated with optimal outcomes for t:slim x2 with control-iq technology in real-world clinical care. Diabetes Technology & Therapeutics, 25(12), 877-882. PMID: 37751154.

Moscoso-Vasquez, M., Colmegna, P., Barnett, C., Fuller, M., Koravi, C. L., Brown, S. A., DeBoer, M. D., and Breton, M. D. (2024). Evaluation of an automated priming bolus for improving prandial glucose control in full closed loop delivery. Diabetes Technology and Therapeutics.

Moscoso-Vasquez, M., Garcia-Tirado, J., Colmegna, P., and Breton, M. (2023). Reshaping model predictive control penalties for an automated insulin delivery system in type 1 diabetes. IFAC-Papers On-Line, 56(2), 9660-9665.

Patek, S. D., Lv, D., Ortiz, E. A., C., H. K., Kulkarni, S., Zhang, Q., and Breton, M. D. (2016). Empirical representation of blood glucose variability in a compartmental model.

Resalat, N., Hilts, W., Youssef, J. E., Tyler, N., Castle, J. R., and Jacobs, P. G. (2019). Adaptive control of an artificial pancreas using model identification, adaptive postprandial insulin delivery, and heart rate and accelerometry as control inputs. Journal of Diabetes Science and Technology, 13, 1044-1053.

Shi, D., Dassau, E., and Doyle, F. J. (2019). Adaptive zone model predictive control of artificial pancreas based on glucose- and velocity-dependent control penalties. IEEE Transactions on Biomedical Engineering, 66, 1045-1054.

Villa-Tamayo, M. F., C, J. L. D., Moscoso-Vasquez, M., Castillo, A., Garcia-Tirado, J., Colmegna, P., and Breton, M. (2022). Model personalization in an advanced automated insulin delivery system: An in-silico exploration. In 2022 10th International Conference on Systems and Control (ICSC 2022), 588-593.

Visentin, R., Campos-Nanez, E., Schiavon, M., Lv, D., Vettoretti, M., Breton, M., Kovatchev, B. P., Man, C. D., and Cobelli, C. (2018). The UVA/Padova type 1 Diabetes Simulator goes from single meal to single day. Journal of Diabetes Science and Technology, 12.

Wiegand, S., Raile, K., Reinehr, T., Hofer, S., Nake, A., Rabl, W., and Holl, R. W. (2008). Daily insulin requirement of children and adolescents with type 1 diabetes: effect of age, gender, body mass index and mode of therapy. European Journal of Endocrinology, 158(4), 543-549.

American Diabetes Association (2024). 7. Diabetes Technology: Standards of care in diabetes—2025. Diabetes Care, 48(Supplement1), S146-S166.

Biester, T., Berget, C., Boughton, C., Cudizio, L., Ekhlaspour, L., Hilliard, M. E., Reddy, L., Sap Ngo Um, S., Schoelwer, M., Sherr, J. L., and Dovc, K. (2024). Ispad clinical practice consensus guidelines 2024: Diabetes technologies: Insulin delivery. Hormone Research in Paediatrics, 1-34.

Castillo, A., Villa-Tamayo, M. F., Pryor, E., Garcia-Tirado, J., Colmegna, P., and Breton, M. (2023). Deep neural network architectures for an embedded MPC implementation: Application to an Automated Insulin Delivery system.

In IFAC-PapersOnLine, volume 56, 11521-11526. Elsevier B. V. Diaz, J., Colmegna, P., Pryor, E., and Breton, M. D. (In press). A performance-based adaptation index for automated insulin delivery systems. Journal of Diabetes Science and Technology.

Forlenza, G. P. and Lal, R. A. (2022). Current status and Emerging options for Automated Insulin Delivery Systems. Diabetes technology & therapeutics, 24(5), 362-371.

Garcia-Tirado, J., Colmegna, P., Villard, O., Diaz, J. L., Esquivel-Zuniga, R., Koravi, C. L., Barnett, C. L., Oliveri, M. C., Fuller, M., Brown, S. A., Deboer, M. D., and Breton, M. D. (2023). Assessment of meal anticipation for improving fully automated insulin delivery in adults with type 1 diabetes. Diabetes Care, 46, 1652-1658. DCLP6.

Garcia-Tirado, J., Lv, D., Corbett, J. P., Colmegna, P., and Breton, M. D. (2021). Advanced hybrid artificial pancreas system improves on unannounced meal response—in silico comparison to currently available system. Computer Methods and Programs in Biomedicine, 211.

Gondhalekar, R., Dassau, E., and Doyle, F. J. (2016). Periodic zone-mpc with asymmetric costs for outpatient-ready safety of an artificial pancreas to treat type 1 diabetes. Automatica, 71, 237-246.

Heusden, K. V., Dassau, E., Zisser, H. C., Seborg, D. E., and Doyle, F. J. (2012). Control-relevant models for glucose control using a priori patient characteristics. IEEE Transactions on Biomedical Engineering, 59, 1839-1849.

Hinshaw, L., Dalla Man, C., Nandy, D. K., Saad, A., Bharucha, A. E., Levine, J. A., Rizza, R. A., Basu, R., Carter, R. E., Cobelli, C., et al. (2013). Diurnal pattern of insulin action in type 1 diabetes: implications for a closed loop system. Diabetes, 62(7), 2223-2229.

Hoyos, J. D., Villa-Tamayo, M. F., Builes-Montano, C. E., Ramirez-Rincon, A., Godoy, J. L., Garcia-Tirado, J., and Rivadeneira, P. S. (2021). Identifiability of control-oriented glucose-insulin linear models: Review and analysis. IEEE Access, 9, 69173-69188.

King, A. B., Kuroda, A., Matsuhisa, M., and Hobbs, T. (2016). A review of insulin-dosing formulas for continuous subcutaneous insulin infusion (csii) for adults with type 1 diabetes. Current diabetes reports, 16, 1-8.

Kovatchev, B., Castillo, A., Pryor, E., Kollar, L. L., Barnett, C. L., DeBoer, M. D., and Brown, S. A. (2024). Neural-net artificial pancreas: A randomized crossover trial of a first-in-class automated insulin delivery algorithm. Diabetes Technology and Therapeutics, 26, 375-382.

Messer, L. H. and Breton, M. D. (2023). Therapy settings associated with optimal outcomes for t:slim x2 with control-iq technology in real-world clinical care. Diabetes Technology & Therapeutics, 25(12), 877-882. PMID: 37751154.

Moscoso-Vasquez, M., Colmegna, P., Barnett, C., Fuller, M., Koravi, C. L., Brown, S. A., DeBoer, M. D., and Breton, M. D. (2024). Evaluation of an automated priming bolus for improving prandial glucose control in full closed loop delivery. Diabetes Technology and Therapeutics.

Moscoso-Vasquez, M., Garcia-Tirado, J., Colmegna, P., and Breton, M. (2023). Reshaping model predictive control penalties for an automated insulin delivery system in type 1 diabetes. IFAC-Papers On-Line, 56(2), 9660-9665.

Patek, S. D., Lv, D., Ortiz, E. A., C., H. K., Kulkarni, S., Zhang, Q., and Breton, M. D. (2016). Empirical representation of blood glucose variability in a compartmental model.

Resalat, N., Hilts, W., Youssef, J. E., Tyler, N., Castle, J. R., and Jacobs, P. G. (2019). Adaptive control of an artificial pancreas using model identification, adaptive postprandial insulin delivery, and heart rate and accelerometry as control inputs. Journal of Diabetes Science and Technology, 13, 1044-1053.

Shi, D., Dassau, E., and Doyle, F. J. (2019). Adaptive zone model predictive control of artificial pancreas based on glucose-and velocity-dependent control penalties. IEEE Transactions on Biomedical Engineering, 66, 1045-1054.

Villa-Tamayo, M. F., C, J. L. D., Moscoso-Vasquez, M., Castillo, A., Garcia-Tirado, J., Colmegna, P., and Breton, M. (2022). Model personalization in an advanced automated insulin delivery system: An in-silico exploration. In 2022 10th International Conference on Systems and Control (ICSC 2022), 588-593.

Visentin, R., Campos-Nanez, E., Schiavon, M., Lv, D., Vettoretti, M., Breton, M., Kovatchev, B. P., Man, C. D., and Cobelli, C. (2018). The UVA/Padova type 1 Diabetes Simulator goes from single meal to single day. Journal of Diabetes Science and Technology, 12.

Wiegand, S., Raile, K., Reinehr, T., Hofer, S., Nake, A., Rabl, W., and Holl, R. W. (2008). Daily insulin requirement of children and adolescents with type 1 diabetes: effect of age, gender, body mass index and mode of therapy. European Journal of Endocrinology, 158(4), 543-549.

1. A computer program product for adjusting automated insulin delivery to a recipient individual, comprising:
a non-transitory computer usable medium having computer readable program code embodied therein, wherein the computer readable program code comprises instructions for implementing a method with a processor connected to computer memory, the method comprising steps of:
accessing population-level insulin data used by an automated insulin delivery device to determine insulin doses for the recipient individual having an individual insulin dose history;
selecting a scaling factor (θ) from the population-level data and calculating, from the population-level data, nominal insulin doses for a nominal individual characterized as a virtual subject in the computer memory;
using the scaling factor (θ) to generate a virtual insulin history for the nominal individual and storing the virtual insulin history for the nominal individual in the computer memory;
calculating a ratio, gamma (γ), equal to the scaling factor (θ indv) for the recipient individual divided by the scaling factor (θ nominal) for the nominal individual;
calculating a standardized insulin dose for the recipient individual by scaling the individual insulin dose history with gamma (γ);
applying the standardized insulin dose to the automated insulin delivery device and calculating an insulin dose recommendation for the recipient individual;
re-scaling the insulin dose recommendation with the ratio, gamma (γ), and calculating a final dose of insulin delivery to the recipient individual.

2. The computer program product of claim 1, wherein the ratio, gamma (γ), corresponds to relative insulin needs of the recipient individual compared to the nominal individual.

3. The computer program product of claim 2, wherein the relative insulin needs scale linearly between the recipient individual and the nominal individual.

4. The computer program product of claim 1, wherein the virtual insulin history for the nominal individual exhibits a linear relationship within the population level data.

5. The computer program product of claim 1, further comprising selecting an automated insulin delivery parameter used by an automated insulin delivery algorithm and scaling the selected parameter with the scaling factor (θ).

6. The computer program product of claim 5, wherein the automated insulin delivery parameter is one of a correction factor (CF), a total daily insulin (TDI), or a model insulin sensitivity factor (Si).

7. The computer program product of claim 1, wherein the method is performed in a closed loop process.

8. The computer program product of claim 1, wherein the method is performed without meal announcement data added to the automated insulin delivery system.

9. The computer program product of claim 1, wherein population level data comprises test data from a Subcutaneous Oral Glucose Minimal Model (SOGMM).

10. The computer program product of claim 1, wherein the method is operated by a controller configured to modify operations according to the individual's estimated basal rate set as ub=TDI/48.

11. A computer implemented method for providing an automated insulin delivery system with personalization of gain and operation point with a non-transitory computer usable medium having computer readable program code embodied therein, wherein the computer readable program code comprises instructions for implementing a method with a processor connected to computer memory, the method comprising steps of:
accessing population-level insulin data used by an automated insulin delivery device to determine insulin doses for the recipient individual having an individual insulin dose history;
selecting a scaling factor (θ) from the population-level data and calculating, from the population-level data, nominal insulin doses for a nominal individual characterized as a virtual subject in the computer memory;
using the scaling factor (θ) to generate a virtual insulin history for the nominal individual and storing the virtual insulin history for the nominal individual in the computer memory;
calculating a ratio, gamma (γ), equal to the scaling factor (θ indv) for the recipient individual divided by the scaling factor (θ nominal) for the nominal individual;
calculating a standardized insulin dose for the recipient individual by scaling the individual insulin dose history with gamma (γ);
applying the standardized insulin dose to the automated insulin delivery device and calculating an insulin dose recommendation for the recipient individual;
re-scaling the insulin dose recommendation with the ratio, gamma (γ), and calculating a final dose of insulin delivery to the recipient individual.
